(19) United States
(12) Patent Application Publication
(10) Pub. No.: US 2013/0109014 A1
(43) Pub. Date: May 2, 2013

(54) AUTOMATIC SYSTEM FOR DETECTION AND IDENTIFICATION OF ISOLATED CELLS FROM BLOOD OR TISSUE

(71) Applicants: Triantafyllos TAFAS, Rocky Hill, CT (US); Michael KILPATRICK, West Hartford, CT (US); Petros TSIPOURAS, Madison, CT (US)

(72) Inventors: Triantafyllos TAFAS, Rocky Hill, CT (US); Michael KILPATRICK, West Hartford, CT (US); Petros TSIPOURAS, Madison, CT (US)

(73) Assignee: IKONISYS, INC., NEW HAVEN, CT (US)

(21) Appl. No.: 13/482,762

(22) Filed: May 29, 2012

Related U.S. Application Data
(63) Continuation of application No. 12/486,646, filed on Jun. 17, 2009, now abandoned.
Application No. 12/486,646 is a division of application No. 11/685,740, filed on Mar. 13, 2007, now abandoned.
(60) Provisional application No. 60/865,796, filed on Nov. 14, 2006.
(60) Provisional application No. 60/781,888, filed on Mar. 13, 2006.

(51) Int. Cl. C12Q 1/68

(52) U.S. Cl. CPC C12Q 1/6881
USPC 435/6.11

(57) ABSTRACT

A non-invasive method for determining the developmental age of a fetus or detecting cancer cells in a sample is provided. The method utilizes, for example, a sample of blood from a pregnant female and telomeric nucleic acid probes.

### CROSS-REFERENCE TO RELATED APPLICATION

This application is a continuation of copending application U.S. patent application Ser. No. 12/486,646, filed Jun. 17, 2009 which is a divisional application of U.S. patent application Ser. No. 11/685,740, filed Mar. 13, 2007 which claims benefit under 35 U.S.C. §119(e) from U.S. Provisional Applications Ser. Nos. 60/865,796, filed on Nov. 14, 2006 and 60/781,888, filed on Mar. 13, 2006.

### BACKGROUND

All references cited in this specification, and their references, are incorporated by reference herein where appropriate for teachings of additional or alternative details, features, and/or technical background.

1. Field of the Invention

Disclosed herein in embodiments are non-invasive methods for the determination of fetal parameters in a pregnant female and for detecting differentiated cells in a cell population based on telomeric markers. In one embodiment, a diagnosis is made based upon fetal cells isolated from a sample of maternal blood, using the fetal telomeric structure as an identifier of fetal cells.

2. Description of the Related Art

Telomeres are structural components of the ends of chromosomes and are formed by a specialized DNA-protein complex (Blackburn E. H. 1994 Cell June 3;77(5):621-3), which contain noncoding DNA repeats and are essential for chromosomal stability and senescence of cells. Telomeric DNA consists of G-rich hexanucleotide repeats TTAGGG in vertebrate cells (Moyzis et al. 1988 Proc. Natl. Acad. Sci. USA 85: 6622-6626) and is folded by telomere binding proteins into a loop structure (Griffith et al. 1999 Mammalian Telomers end in a large duplex loop. Cell, May, 14; 97(4): 503-514). Telomeres appear to maintain the integrity of chromosomes by protecting against inappropriate recombination and random end-to-end fusions of chromosomes and in preventing incomplete DNA replication of chromosomes in cell division.

The loss of telomere repeats associated with replication of human somatic cells in culture has demonstrated that telomeres serve as “mitotic clocks,” cellular senescence and aging of organisms, and that the telomere length is a biomarker of replicative history of the cells that is modified by genetic factors and sex. For example, in humans, telomere length of replicating somatic cells is inversely related to donor age (Vaziri et al. 1993 Loss of Telomeric DNA during Aging of Normal and Trisomy 21 Human Lymphocytes, Amer. J. Hum. Genet., 52:661-667; Slagboom et al. 1994 Genetic determination of telomere size in humans: a twin study of three age groups, Amer. J. Hum. Genet. November, 55(5): 876-882, and Okuda et al. 2000 Telomere attrition of the human abdominal aorta: . . . Atherosclerosis October; 152(2): 391-398); highly variable among donors of the same age (Vaziri et al. 1993, ibid; Slagboom et al. 1994, ibid, and Okuda et al. 2000 ibid); highly heritable (Slagboom et al. 1994, ibid, and Jeanclos et al., 2000 Telomere length inversely correlates with pulse pressure . . . Hypertension 36: 195-200) and longer in women than in men (Jeanclos et al. 2000 Hypertension 36: 195-200, and Benetos et al. 2001 Telomere length as an indicator of biological aging: . . . Hypertension 37: 381-385). Telomere length has been used as a tool for the analysis of cell division and to analyze lineage or precursor-product relationships and rates of cell division (Rufer et al. 1998 Telomere length dynamics in human lymphocyte subpopulations measured by flow cytometry, Nat. Biotechnol.; Rufer et al. 1999 Telomere fluorescence measurement in granulocytes and T lymphocyte subset point to a high turnover of hematopoietic stem cells and memory T cells in early childhood, J. Exp. Med., July 19; 190(2): 157-167, and Son et al. 2000 Lineage-specific telomere shortening and unaltered capacity for telomerase expression in human T and B lymphocytes with age, J. Immunol. 165: 1191-1196). Age-related telomere shortening in various subpopulations of blood cells in humans has been demonstrated by fluorescent in situ hybridization (Baerrlocher et al. Telomere length measurement by fluorescence in situ hybridization and flow cytometry: tips and pitfalls, Cytometry 47:89-99, 2002).

Upon cell transformation, when telomeres shorten exaggeratedly, chromosome extremities become unstable and fuse to each other, resulting in breakage-fusion-bridge cycles (BFB). Telomere shortening has been seen as a prevalent alteration in a number of cancers, including prostatic, pancreatic, and breast cancer lesions (Meeker et al., 2004 Telomere Length Abnormalities Occur Early in the Initiation of Epithelial Carcinogenesis,” Clinical Cancer Research, 10: 3317-3326). However, long telomere length has been linked to poor prognosis of other cancers such as colorectal cancer (Cancer 2006, 106: 541-551). High dose chemotherapy has been found in some cancers, for example, breast cancer, to accelerate telomere length loss in certain cells, such as haematopoietic stem cells (Schröder et al., “Telomere length in breast cancer patients before and after chemotherapy with or without stem cell transportation,” 2001 British J. of Cancer 84: 1348-1353).

Telomere length in humans during intrauterine life has been demonstrated to be highly synchronized in that it is similar among tissues of the same fetus, but can be variable among fetuses (Youngren et al. 1998 Synchrony in telomere length of the human fetus, Hum. Genet. June, 102(6): 640-643). However, there appears to be no differences in the telomere length between male and female newborns (at birth) (Okuda et al. 2002 Telomere length in the newborn, September, 52(3): 377-381).

Present methods for fetal diagnosis in utero include invasive techniques such as amniocentesis in which fluid is removed from the amniotic sac of the developing fetus. While this procedure is widely used, in certain situations, it can lead to fetal damage and/or an abortion. Repeated sonograms of the fetus during development has been a concern for mothers to be due to the repeated exposure of the fetus to ultrasonic radiation for undetermined periods of time. Some believe the ultrasonic radiation used to image the fetus during a sonogram may potentially cause problems in later life. Therefore, new and more accurate noninvasive techniques for fetal diagnosis are needed to protect the pregnant female and fetus.

Numerous methods are known for determining telomere length including measurement by Southern blot (Satillo-Pineiro et al., “Telomerase activity and telomere length in primary and metastatic tumors from pediatric bone cancer patients, ” 2004 Pediatric Res. 55(2): 231-235), and fluorescent in situ hybridization dot counting (Schulze et al. “Telomere Length Measurements” April 2000, Proc. First Euroconference on Quantitative Molecular Epiginetics) alone or in conjunction with flow cytometry (Suleman, S. “Telomere Length Analysis as a Novel Diagnostic Test for Bladder Cancer,” Enq. J. Interdisciplinary Studies for High School Students, 1(1): 1-5, 2003). Such systems are typically set up to detect a difference in telomere length between two pre-selected cell populations, thereby failing to provide a robust system for allowing differentiation of a rare cancer cell from other normal cells in its milieu.

Many methods are known to aid in the microscopic analysis of samples. For example, without limitation, it is known that certain dyes have an affinity for certain cellular structures. Such dyes may therefore be used to aid in analysis by helping to further elucidate such structures.

Fluorescence microscopy of cells and tissues is well known in the art. Methods have been developed to image fluorescently-stained cells in a microscope and extract information about the spatial distribution and temporal changes occurring in these cells. Some of these methods and their applications are described in an article by Taylor, et al. in American Scientist 80 (1992), p. 322-335. These methods have been designed and optimized for the preparation of a few specimens for high spatial and temporal resolution imaging measurements of distribution, amount and biochemical environment of the fluorescent reporter molecules in the cells. Detection of fluorescent signals may be by way of an epifluorescent microscope which uses emitted fluorescent light to form an image. The excitation light of a epifluorescence microscope is used to excite a fluorescent tag in the sample causing the fluorescent tag to emit fluorescent light. The advantage of an epifluorescence microscope is that the sample may be prepared such that the fluorescent molecules are preferentially attached to the biological structures of interest thereby allowing identification of such biological structures of interest.

The acronym “FISH” references a technique that uses chromophore tags (fluorophores) that emit a secondary signal if illuminated with an excitation light to detect a chromosomal structure. FISH uses fluorescent probes which bind only to those parts of the chromosome with which they show a high degree of sequence similarity. Such tags may be directed to specific chromosomes and specific chromosome regions. The probe has to be long enough to hybridize specifically to its target (and not to similar sequences in the genome), but not too large to impede the hybridization process, and it should be tagged directly with fluorophores. This can be done in various ways, for example nick translation or PCR using tagged nucleotides. If signal amplification is necessary to exceed the detection threshold of the microscope (which depends on many factors such as probe labelling efficiency, the kind of probe and the fluorescent dye), secondary fluorescent tagged antibodies or streptavidin are bound to the tag molecules, thus amplifying the signal.

The FISH technique may be used for identifying chromosomal abnormalities and gene mapping. For example, a FISH probe to chromosome 21 permits one to “fish” for cells with trisomy 21, an extra chromosome 21, the cause of Down syndrome. FISH kits comprising multicolor DNA probes are commercially available.

Diagnostic FISH dot counting has been conventionally performed manually, by a skilled microscopist. In addition to correctly identifying the dot and it's color, other size and shape characteristics must be categorized to correctly identify the chromosomal condition.

The analysis is made more difficult by the time constraints imposed by the phenomena. The microscopist, therefore, must be trained to perform the examination. Even under the best conditions, the process has proven to be tedious, lengthy and subject to human error.

The application of automated microscopy has the potential to overcome many of the shortcomings of the manual approach. The automatic microscope can reliably identify the fluorescent dots in a sample, accurately determine their color, categorize them based on shape and size, and perform the summary analysis necessary to determine the presence or absence of the targeted condition without the inevitable subjective factors introduced by a human operator all in a timely manner.

No techniques and/or information are available presently to determine telomere length in developing fetuses. Methods for detecting cancer cells from other cells in a sample without the need for laborious separation of cells or labor intensive categorization of the cells is also lacking. We provide herewith methods for automatically determining telomere length following fluorescence in situ hybridization.

### SUMMARY

In an embodiment of the invention, there is disclosed a method for diagnosing fetal cells, said method comprising the steps of isolating a sample of blood from a pregnant female; isolating fetal cells from said blood sample; and identifying the fetal cells by determining telomeric length using telomeric nucleic acid probes designed to hybridize the ends of the telomere.

In one embodiment, there is provided a method for diagnosing fetal cells using the material blood at any stage of pregnancy. The method comprises: isolating a sample of blood from a pregnant female; isolating fetal cells from said blood sample; and identifying said fetal cells by in-situ hybridization techniques using telomeric nucleic acid probes. Parameters of the fetal cell identified may be measured, for example, allowing one to determine the developmental age of a fetus.

In another embodiment, there is disclosed a method for detecting a cancer cell distributed among a plurality of normal cells, comprising: obtaining a tissue sample from a patient; hybridizing chromosomal DNA of cells in said tissue sample with nucleic acid probes comprising telomeric DNA, RNA and/or PNA using fluorescent in situ hybridization (FISH) conditions to obtain a treated sample; and analyzing said treated sample on an automated microscope system operatively programmed to:
automatically search optical fields with respect to said treated sample to detect fluorescent signals indicative of said nucleic acid probes binding to chromosomal telomere DNA to identify telomere;
identifying cells having a distinctly different chromosomal telomeric DNA from other cells in the treated sample;
comparing said cells identified to having a distinctly different chromosomal telomeric DNA against a predetermined telomeric DNA binding standard indicative of a cell in a cancerous state; and outputting information pertaining to whether a cancerous state is detected or not.

### DETAILED DESCRIPTION

In embodiments illustrated herein, there is disclosed the detection and measurement/quantification of telomere length using fluorescent in situ hybridization (FISH) methods and systems for detecting and monitoring the presence of fluorescent signals with the employment of automated detection microscopy.

In one embodiment, there is provided a method for diagnosing fetal cells, the method comprising the steps of: (a) isolating a sample of blood from a pregnant female; (b) isolating fetal cells from the blood sample; and (c) identifying the fetal cellsby determining the telomeric length using telomeric nucleic acid probes designed to hybridize the ends of the telomere. When the developmental age of a fetus from which the fetal cell issues is to be adjudged, one may determine the same by looking at telomeric length.

The detection and quantification may be used as an additional fetal cell marker in identifying fetal cells in a sample of maternal blood. In one embodiment, the chromosomal telomere length of cells can be combined with the detection of fetal hemoglobin. An in situ hybridization technique may be used and the detection of nucleic acid probe hybridization to chromosomal DNA in the sample cells may be enhanced by using a computerized robotic microscope. In this embodiment, the detection of fetal cells in maternal blood can be used to significantly increase the efficiency of identifying nucleated red blood cells in maternal blood samples.

The identification and determination of fetal cells may also be made by measurements of the telomeric length of the chromosomal ends of nucleated fetal cells isolated from a sample of maternal blood using at least one specific telomeric DNA probe labeled with a fluorescent label and a fetal specific detection probe such as fetal hemoglobin gamma.

Quantitation of telomere length using in situ hybridization can be used as a marker to identify fetal cells within a population of adult cells. Fetal cell detection in the maternal circulation is a very desirable, and poses a low risk method for successful prenatal diagnosis. Measurements of the telomeres can be made using a rapid detection system and analyzed under a fluorescent computerized robotic microscope. Data obtained from the maternal blood sample is compared to control sample data.

In an embodiment, the identification of fetal cells and in particular nucleated red blood cells of fetal origin is accomplished using distinguishable characters present in the cells such as the identification of the existence of a nucleus in the cells and the presence of hemoglobin gamma as compared to hemoglobin alpha present in mature maternal red blood cells.

Methods of the invention may detect fetal cells even when the donor blood sample is from an anemic adult individual. In this embodiment, certain anemic patients whose blood cells can express significant levels fetal hemoglobin can be screened with the telomeric probes to identify very rare fetal nRBCs among a vast population of maternal cells.

In another embodiment, there is disclosed a method for detecting rare cancer cells distributed among a plurality of normal cells from a sample tissue, for example, blood. After the tissue is obtained from a patient, the tissue sample is processed using a fluorescent in situ hybridization procedure for detecting differences in chromosomal telomeric length of the cells in the sample tissue. The technique uses telomeric nucleic acid probes which hybridize to chromosomal DNA of cells in the tissue sample. The nucleic acid probes comprising telomeric DNA, RNA and/or PNA tagged or labeled with a fluorescent dye of a selected color are used in the hybridization techniques. After hybridization, the sample is analyzed using an automated microscope system comprising a computer program which automatically searches optical fields with respect to said sample to detect fluorescent signals. Fluorescent signals obtained from the sample are indicative of the nucleic acid probes hybridization with the chromosomal telomere DNA and identify the presence of telomere in the sample cells. The intensity of the fluorescent probe may be quantified as directly proportional to the length of telomere DNA present in the cells. Cells having a distinctly different chromosomal telomeric DNA length from other cells in the treated sample when compared to a predetermined standard can be identify as being, for example, in a cancerous state. The automated microscope system can output information pertaining to whether a cancerous state is detected.

### STATEMENT REGARDING PREFERRED EMBODIMENTS

While the invention has been particularly shown and described with reference to particular embodiments, it will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1-3. (canceled)

4. The method of claim 15, wherein the DNA probe is directly or indirectly labeled with a fluorescent compound of a select color.

5-11. (canceled)

12. The method for diagnosing fetal cells of claim 15, wherein said labels possess distinguishable spectral characteristics.

13. The method for diagnosing fetal cells of claim 15, wherein said samples are further treated with standard histological stains comprising DAPI, Hemotoxylin, Eosin, toluidine blue, Wright's stain.

14. (canceled)

15. An automated method for detecting cancer cells with at least one abnormal chromosomal component distributed among a plurality of normal cells, comprising:
obtaining on a substrate a tissue or blood sample from a patient;
hybridizing chromosomal DNA of the cells in said tissue or blood sample to nucleic acid probes comprising telomere DNA, RNA and/or PNA distinguishably color labeled fluorescent in situ hybridization (FISH) conditions to obtain a treated sample;
analyzing simultaneously said distinguishably color labeled fluorescent cells with said at least one abnormal chromosomal component on a high-throughput robotic digital microscopy platform programmed to automatically:
(i) search optical fields on said substrate with respect to said treated sample to detect fluorescent signals indicative of said nucleic acid probes binding to chromosomal telomere DNA to identify a telomere;
(ii) locate said one or more fluorescent distinguishably color labeled cells at low magnification;
(iii) identify cells having a distinctly different chromosomal telomere DNA length from other cells in the treated sample on the basis of a predetermined telomere DNA binding standard indicative of a cell in a cancerous state;
(iv) verify a type of cancerous cell bearing said at least one said distinguishably color labeled probe at high magnification;
(v) count normal cells and said type of cancerous cells in a field;
(vi) provide a representation of said type of cancerous cells in said normal cell count; and
(vii) outputting information pertaining to a ratio of said labeled cells wherein a cancerous state is detected.
